(19) United States
(12) Patent Application Publication
(10) Pub. No.: US 2012/0150198 A1
(43) Pub. Date: Jun. 14, 2012

(54) Ergonomically Correct Needle Holder

(76) Inventor: Alex Galyuk, West Hollywood, CA (US)

(21) Appl. No.: 12/968,169

(22) Filed: Dec. 14, 2010

(51) Int. Cl. A61B 17/04

(52) U.S. Cl. 606/147

(57) ABSTRACT

An ergonomically correct suture needle holder is disclosed. The ergonomically correct suture needle holder comprises a thumb handle having a thumb spinning grip and a ring finger handle having a ring finger grip with a finger holder extending therewith. The ring finger handle and the thumb handle can be pivoted from an open position to a closed position. The needle holder is maintained in the closed position by engaging a first ratchet locking member protruding from the thumb handle with a second ratchet locking member protruding from the ring finger handle. The suture needle holder facilitates the user to twist the user's wrist without moving the user's hand thereof. The thumb spinning grip is custom sized to accommodate a user's thumb and facilitates a swiveling motion of the user's thumb while engaging in surgical movements for allowing the user to suture comfortably without changing the grip on the needle holder.

### CROSS-REFERENCE TO RELATED APPLICATIONS

Not Applicable.

### STATEMENT REGARDING FEDERALLY SPONSORED RESEARCH AND DEVELOPMENT

Not Applicable.

### FIELD OF THE INVENTION

This invention relates to surgical instruments, and more particularly to an ergonomic suture needle holder adapted for one-hand operation facilitated by a swiveling motion of a user's thumb during suturing.

### DISCUSSION OF RELATED ART

Suturing is a common surgical procedure for repairing wounds and incisions. Suture needle holders are commonly utilized for gripping and suturing inside the skin and inside deep tissue. A conventional suture needle holder may include a pair of movable opposed jaws connected to a pair of handles having finger loops. The finger loops are standard sized which may not fit perfectly and fail to engage in a surgeon's hand making it difficult for the surgeon to grip the needle holder to secure the suture in place. In addition, a conventional needle holder does not allow the surgeon to make a complete twist of his wrist without moving the surgeon's hand. Different kinds of tools for suturing in deep surgical apertures or body cavities have been discussed in the prior arts.

One prior art taught in U.S. Pat. Application No. 20070144013 entitled to Ishida on Jun. 28, 2007 provides an ergonomic hand tool that permits variations in finger relief to accommodate variations in human physiology and a finger handle that aligns the user's fingers into a minimum stress position relative to each other and to the thumb. However, the user's fingers may tend to get stuck inside the finger handles making it uncomfortable to move the fingers during suturing. Further, the tool does not provide any mechanism to lock the finger handles in the closed position.

Another prior art taught in U.S. Pat. No. 5,833,697 issued to Ludwick on Nov. 10, 1998 discloses a surgical instrument for holding a suture needle. The instrument is of a scissors configuration with elongated arms having finger loops at one end and jaws at the other end to grasp a suture needle. This improved instrument has specialized jaws containing a transverse channel that securely holds a suture needle at a right angle to the axis of the needle holder jaws. The width of the transverse channel adjusts to the size of the suture needle by means of a cam and spring loaded camming surface which move a sliding member against the side of a suture needle as the jaws of the needle holder are closed.

However, the finger loops may not firmly engage the user's finger thereby causing slippage of the instrument.

U.S. Pat. No. 4,580,567 issued to Schweitzer on Apr. 8, 1986 disclose a suture needle holder that enables a surgeon to manipulate a suture needle with one hand. A thumb handle of the needle holder has a freely rotatable short roller on one end and a ring finger handle of the holder has a long roller that is journalled within that handle. One end of the long roller bears against the short roller to form a nip that engages the suture needle. The other end of the long roller terminates near the ring finger handle grip to enable the surgeon to rotate the long roller with another finger of the same hand and thereby draw the needle through the nip, repositioning the needle for another stitch. However, the above described suture needle holder causes increased stress on the surgeon's hand.

Therefore, there is a need for an ergonomic suture needle holder that facilitates a swiveling motion of a user's thumb thereby eliminating the necessity for the user to change the grip on the needle holder during suturing. Such a needle holder would utilize a ratchet mechanism to lock finger handles in a closed position thereby maintaining firm gripping pressure on the suture needle. Further, this needle holder would include finger loops that are shaped and sized to engage fingers of all sizes to minimize stress and holder slippage while facilitating better wrist movement. The present invention accomplishes all these objectives.

### SUMMARY OF THE INVENTION

The present invention is an ergonomically correct suture needle. The ergonomically correct suture needle holder comprises a thumb handle having a lower end terminating in a first ratchet locking member and an upper end terminating in a needle holder jaw, and a ring finger handle having a lower end terminating in a ring finger grip and an upper end terminating in the needle holder jaw. The ring finger handle further comprises a second ratchet locking member at a junction of the ring finger grip and the ring finger handle, and a finger holder protruding from the ring finger grip. The thumb handle further comprises a thumb spinning grip attached to the lower end by means of a connecting pin. The ring finger handle is pivotally joined to the thumb handle by means of a pivot pin. The pivot pin facilitates counter rotation of the ring finger handle and the thumb handle about the pivot pin from a closed position to an open position and from the open position to the closed position. The first ratchet locking member releasably engages with the second ratchet locking member to maintain the suture needle holder in the closed position.

The thumb spinning grip may be oval in shape and custom sized to accommodate a user's thumb. The thumb spinning grip facilitates a swiveling motion of the user's thumb while engaging in surgical movements for allowing the user to suture comfortably without changing the grip on the suture needle holder. The user can twist the user's wrist to make a suture without moving the user's hand from the suture needle holder. The finger holder facilitates the user to maintain a secure grip on the suture needle holder and to suture without engaging the thumb spinning grip.

### DESCRIPTION OF THE DRAWINGS

FIG. 1 is a perspective view of the present invention, illustrating an ergonomically correct suture needle holder in a closed position;

FIG. 2 is a perspective view of the present invention, illustrating the ergonomically correct suture needle holder in an open position;

FIG. 3 is a perspective view of the present invention showing a thumb spinning grip slightly angled relative to a thumb handle;

FIG. 4 is a perspective view of the present invention in use, illustrating a twisting motion of the user's wrist in a backward position;

FIG. 5 is a perspective view of the present invention in use, illustrating a twisting motion of the user's wrist in a forward position;

FIG. 6 is a perspective view of the present invention in use, illustrating a ring finger grip and a finger holder facilitating a secure grip when the suture needle holder is in the open position;

FIG. 7 is a perspective view of the present invention in use, illustrating the gripping action when the suture needle holder is in the closed position; and

FIG. 8 is another embodiment of the present invention shown in FIG. 1.

### DETAILED DESCRIPTION OF THE PREFERRED EMBODIMENT

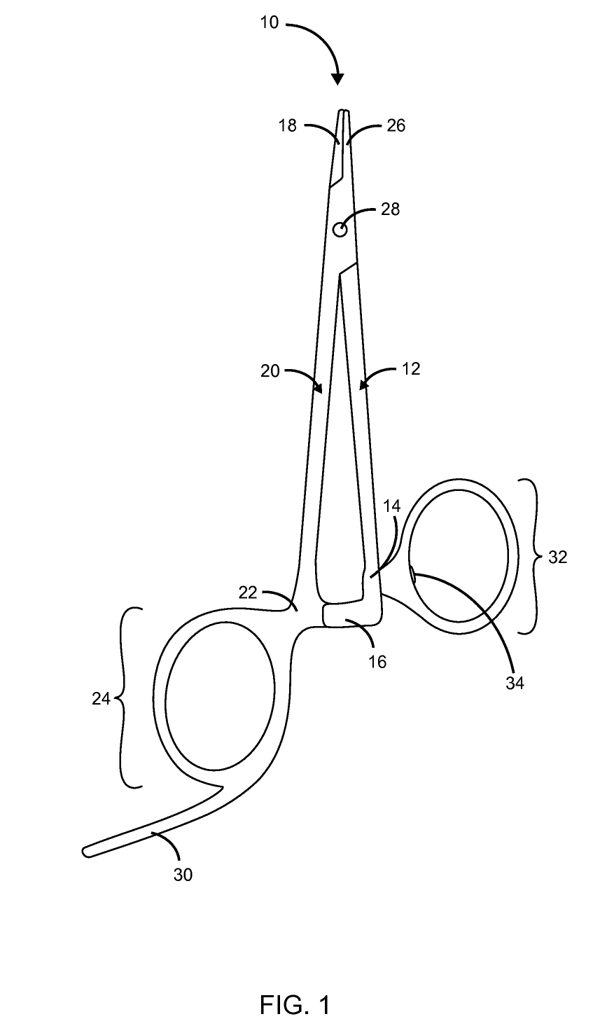

FIG. 1 is a perspective view of the present invention, illustrating an ergonomically correct suture needle holder 10 in a closed position. The ergonomically correct suture needle holder 10 comprises a thumb handle 12 having a lower end 14 terminating in a first ratchet locking member 16 and an upper end 18 terminating in a needle holder jaw (not shown), and a ring finger handle 20 having a lower end 22 terminating in a ring finger grip 24 and an upper end 26 terminating in the needle holder jaw (not shown). The ring finger handle 20 is pivotally joined to the thumb handle 12 by means of a pivot pin 28. The ring finger handle 20 further comprises a second ratchet locking member (not shown) at a junction of the ring finger grip 24 and the ring finger handle 20, and a finger holder 30 protruding from the ring finger grip 24. The thumb handle 12 further comprises a thumb spinning grip 32 attached to the lower end 14 by means of a connecting pin 34. The thumb spinning grip 32 facilitates a swiveling motion of a user's thumb (not shown) while engaging in surgical movements for allowing the user (not shown) to suture comfortably without changing the grip on the ergonomically correct suture needle holder 10.

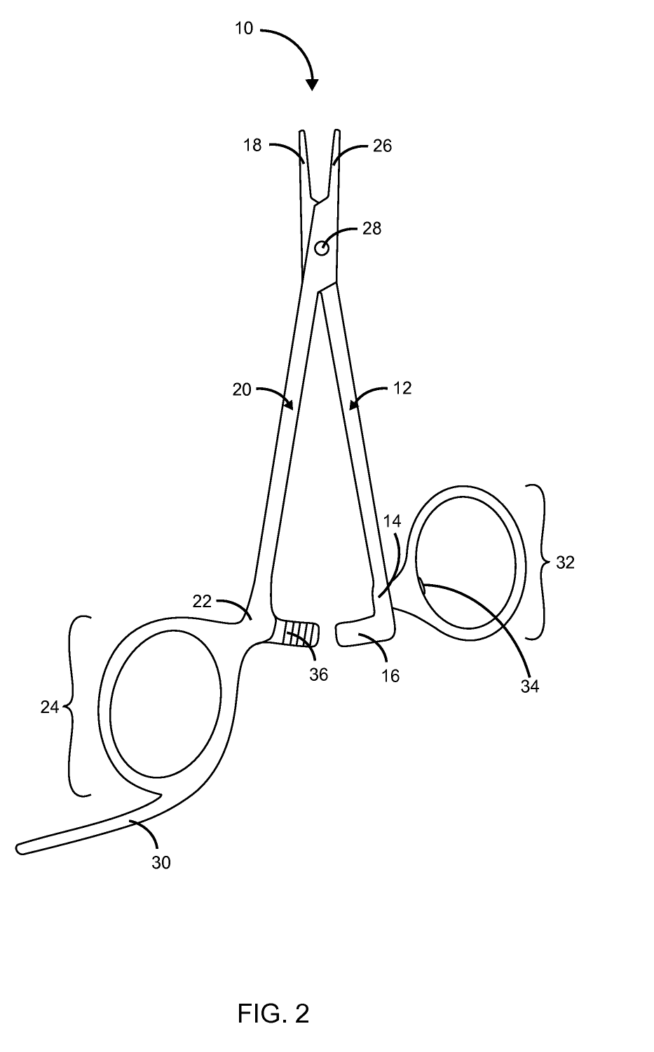

The first ratchet locking member 16 protrudes from the thumb handle 12 towards the ring finger handle 20 and the second ratchet locking member 36 protrudes from the ring finger handle 20 to the thumb handle 12 as shown in FIG. 2. The pivot pin 28 facilitates counter rotation of the ring finger handle 20 and the thumb handle 12 about the pivot pin 28 from the closed position to an open position and from the open position to the closed position.

The first ratchet locking member 16 releasably engages with the second ratchet locking member 36 to maintain the suture needle holder 10 in the closed position. The thumb handle 12 is shorter in length than the ring finger handle 20 providing relative smooth pivoting motion and easy handling of the suture needle holder 10.

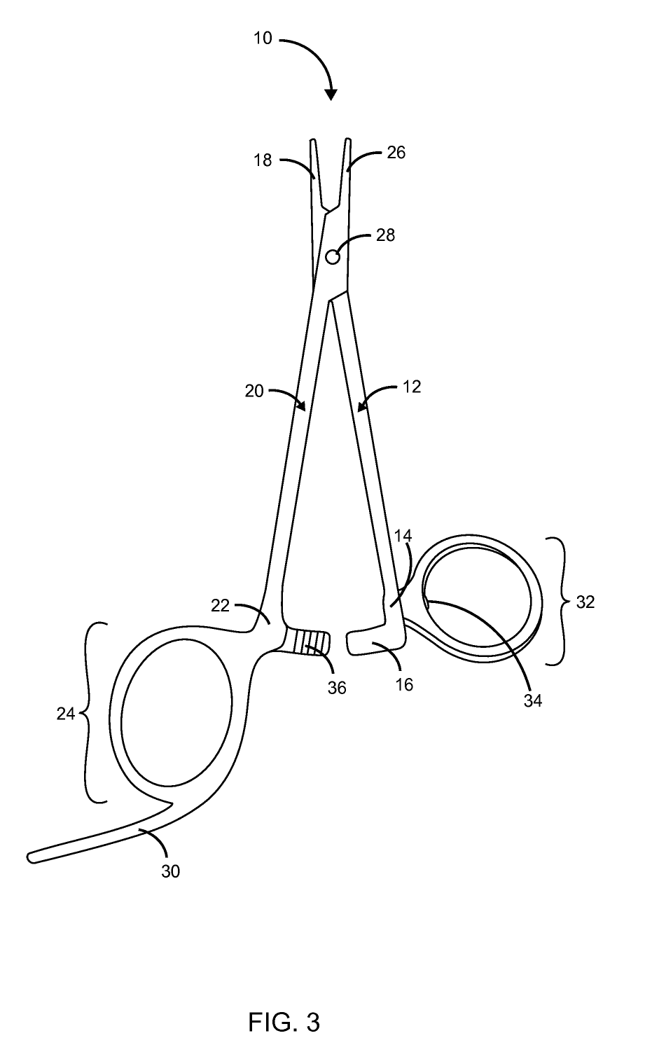

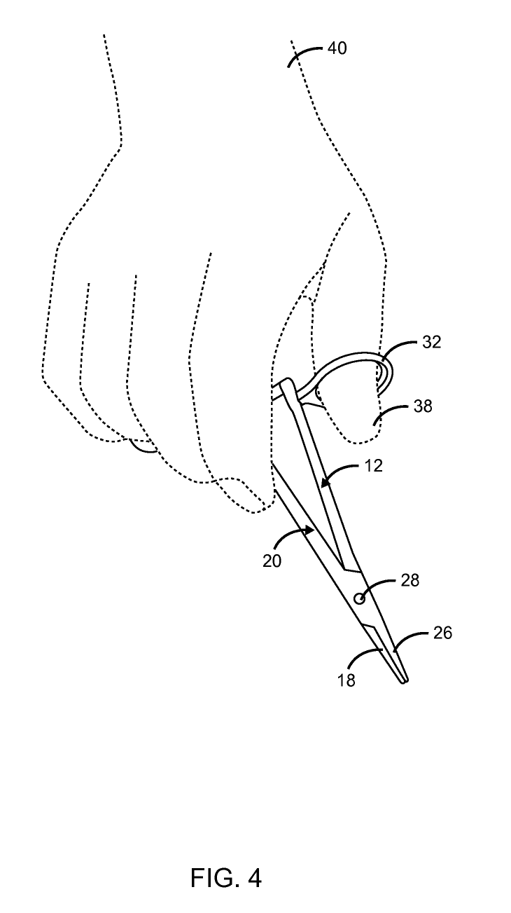

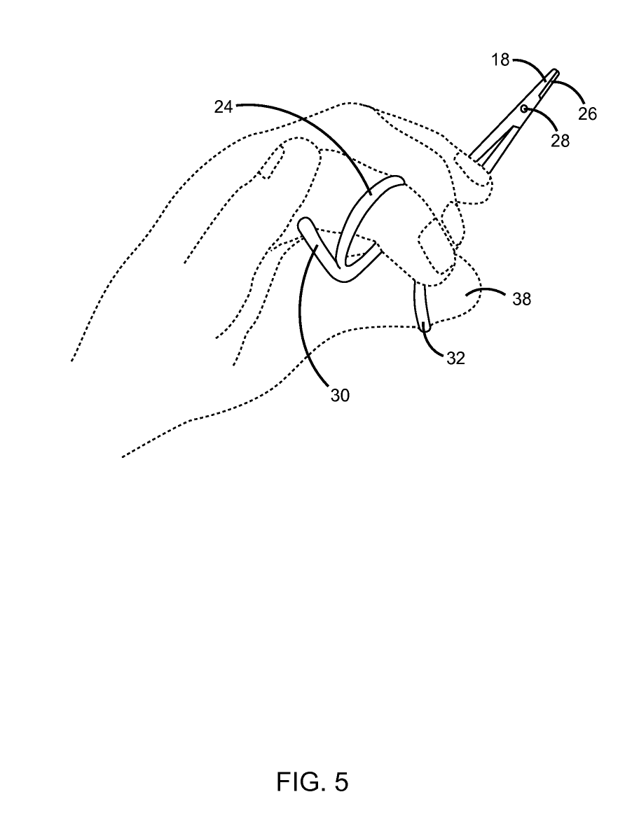

FIG. 3 is a perspective motion of the present invention showing a thumb spinning grip 32 slightly angled relative to the thumb handle 12. The thumb spinning grip 32 may be oval in shape and includes a tapered edge to securely engage the user's thumb 38. The tapered edge allows greater stability and a comfortable and secure fit around the user's thumb 38 thereby facilitating the user (not shown) to achieve maximum control of the suture needle holder 10. The thumb spinning grip 32 may be custom sized to accommodate the user's thumb 38. The user (not shown) can swivel the user's thumb 38 allowing easier suturing motion particularly inside the skin and inside the deep tissue. As illustrated in FIG. 4 and FIG. 5, the user (not shown) can comfortably grip the suture needle holder and twist the user's wrist 40 to make a suture without moving the user's hand (not shown) thereof.

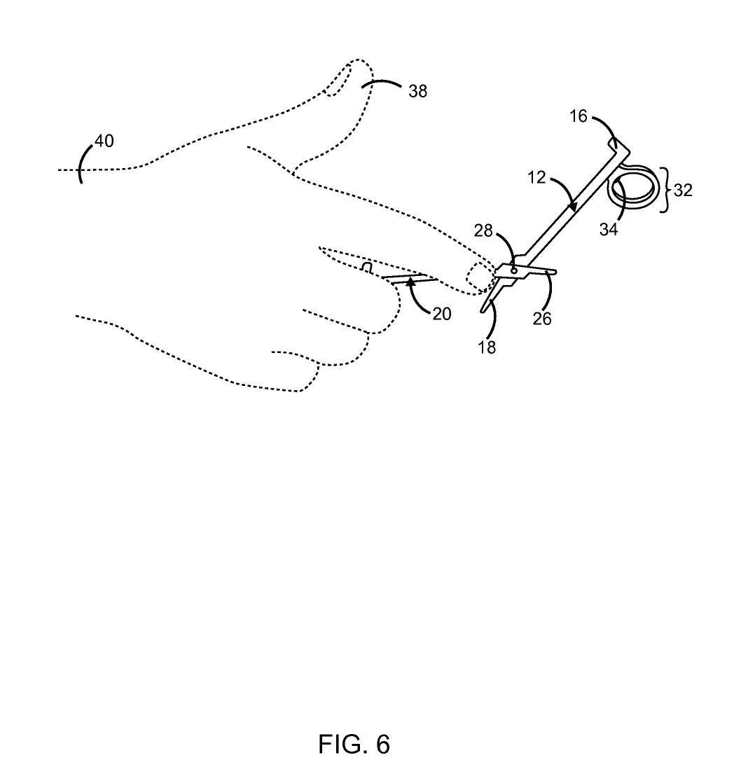

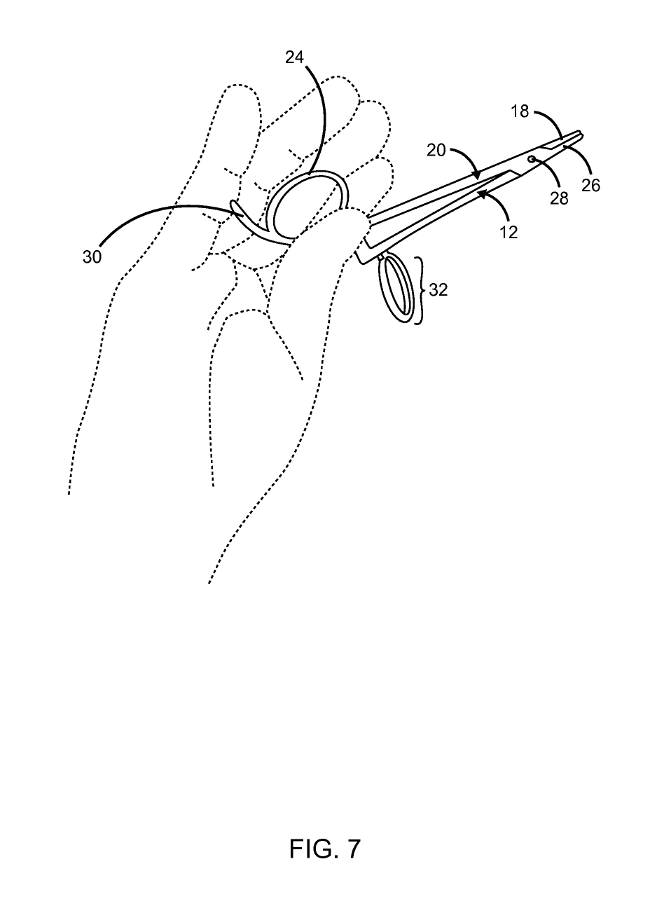

The ring finger grip 24 and the finger holder 30 facilitates the user (not shown) to maintain a secure grip on the suture needle holder 10 in the open position and to suture without engaging the thumb spinning grip 32, as illustrated in FIG. 6 and FIG. 7. The user (not shown) can hold the suture needle holder 10 by engaging the ring finger grip 24 and the finger holder 30 even if the ratchet locking members disengage. Therefore, the user (not shown) does not have to worry about dropping the suture needle holder 10 while engaging in suturing motion.

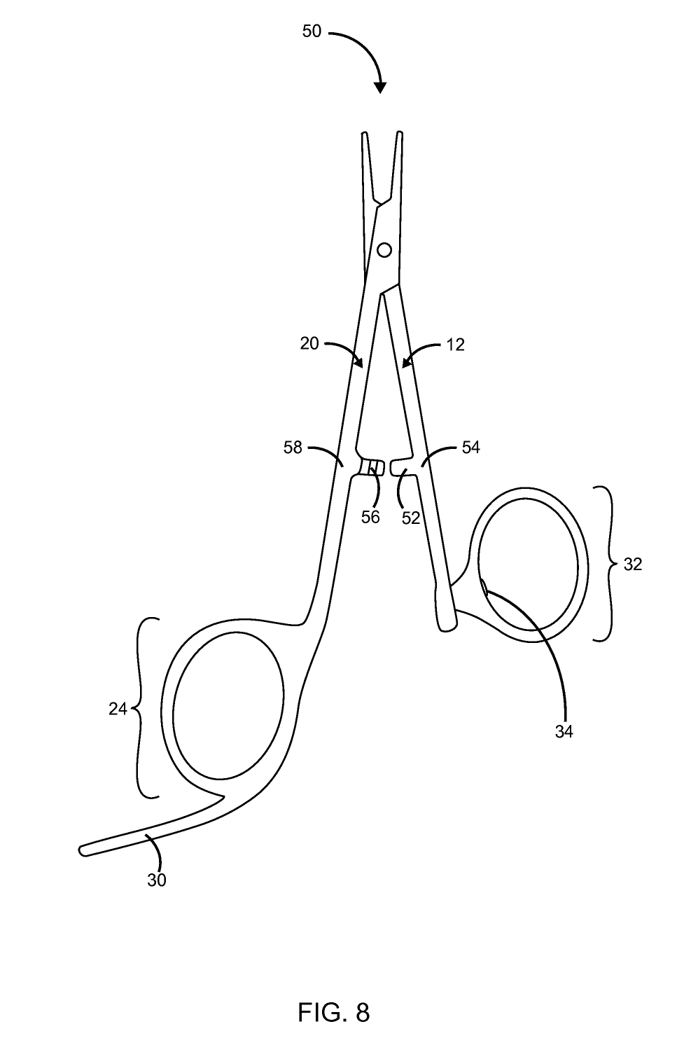

In another embodiment of the present invention, illustrated in FIG. 8, an ergonomically correct suture needle holder 50 comprises the thumb handle 12 and the ring finger handle 20. The thumb handle 12 and the ring finger handle 20 may be pivotally mounted in a scissors configuration. The thumb handle 12 includes a first ratchet locking member 52 protruding from a middle portion 54 towards the ring finger handle 20. The ring finger handle 20 includes a second ratchet locking member 56 protruding from a middle portion 58 towards the thumb handle 12. The first ratchet locking member 42 releasably engages with the second ratchet locking member 46 to hold the suture needle holder 40 in the closed position. The suture needle holder 40 facilitates easier ratcheting mechanism and eliminates the interference of the ratchet locking members ahead the user's ring finger (not shown).

While a particular form of the invention has been illustrated and described, it will be apparent that various modifications can be made without departing from the spirit and scope of the invention. Accordingly, it is not intended that the invention be limited, except as by the appended claims.

What is claimed is:

1. An ergonomically correct suture needle holder comprising:
a thumb handle having a lower end and an upper end, the lower end terminating in a first ratchet locking member and the upper end terminating in a needle holder jaw, the thumb handle including a thumb spinning grip attached to the lower end thereof; and
a ring finger handle having a lower end and an upper end pivotally joined to the thumb handle, the lower end terminating in a ring finger grip and the upper end terminating in the needle holder jaw, the ring finger handle including a second ratchet locking member at a junction of the ring finger grip and the ring finger handle;
whereby the thumb spinning grip facilitating a swiveling motion of a user's thumb while engaging in surgical movements for allowing the user to suture comfortably without changing the grip on the needle holder.

2. The ergonomically correct suture needle holder of claim 1 wherein the thumb spinning grip may be oval in shape.

3. The ergonomically correct suture needle holder of claim 1 wherein the thumb spinning grip may be attached to the lower end by means of a connecting pin.

4. The ergonomically correct suture needle holder of claim 1 wherein the thumb spinning grip includes a tapered edge to securely engage the user's thumb.

5. The ergonomically correct suture needle holder of claim 1 wherein the thumb spinning grip may be custom sized to accommodate the user's thumb.

6. The ergonomically correct suture needle holder of claim 1 wherein the ring finger handle includes a finger holder protruding from the ring finger grip.

7. The ergonomically correct suture needle holder of claim 6 wherein the finger holder facilitates the user to maintain a secure grip on the needle holder and to suture without engaging the thumb spinning grip.

8. The ergonomically correct suture needle holder of claim 1 wherein the ring finger handle is pivotally joined to the thumb handle by means of a pivot pin.

9. The ergonomically correct suture needle holder of claim 8 wherein the pivot pin facilitates counter rotation of the ring finger handle and the thumb handle about the pivot pin from a closed position to an open position and from the open position to the closed position.

10. The ergonomically correct suture needle holder of claim 1 wherein the first ratchet locking member protrudes from the thumb handle towards the ring finger handle and the second ratchet locking member protrudes from the ring finger handle to the thumb handle.

11. The ergonomically correct suture needle holder of claim 1 wherein the first ratchet locking member releasably engages with the second ratchet locking member to hold the suture needle holder in the closed position.
